(19) United States
(12) Patent Application Publication
(10) Pub. No.: US 2008/0160497 A1
(43) Pub. Date: Jul. 3, 2008

(54) Methods and systems for determining mid-value titers

(76) Inventor: Jozef J. P. Nauta, CP Weesp (NL)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/585,577

(22) Filed: Aug. 7, 2006

Related U.S. Application Data
(60) Provisional application No. 60/706,449, filed on Aug. 9, 2005.

(51) Int. Cl. A01N 1/02; G01N 31/00; B01J 19/00; C12M 1/00; G06F 19/00

(52) U.S. Cl. 435/2; 436/18; 422/75; 435/287.2; 702/19

(57) ABSTRACT

The invention provides methods for determining the mid-value titer of a substance in a composition. In some methods the substance comprises at least one anti-hemagglutinin (HA) antibody. The invention also provides systems and computer-readable media for use in association with such methods.

This application claims priority under 35 U.S.C. § 119(e) to Provisional Application No. 60/706,449, filed Aug. 9, 2005, the entire contents of which are hereby incorporated herein by reference.

The present invention generally relates to methods and systems for determining titration values. In some embodiments of the present invention, such methods and systems yield titration values that are more accurate than current methods and systems.

Titration is a generally accepted method for quantifying the concentration of a substance in a composition such as a solution. Some titration assays are based on a series of dilutions and the determination of the dilution at which a certain assay read-out can be detected (or can no longer be detected). As a result, the concentrations existing in-between dilution steps cannot be measured exactly and the assay result only approximates the true titer.

An example of such a measurement is the assay for the determination of anti-hemagglutinin (HA) antibody concentrations, the hemagglutination inhibition (HI) assay. Hemagglutinin has the capacity of binding to erythrocytes resulting in agglutination (hemagglutination), which can be visually detected and thus used as assay read-out. The binding of HA to erythrocytes is inhibited by the addition of serum containing anti-HA antibodies. Thus, the concentration of anti-HA antibodies can be defined as HI titer by incubating serial dilutions of sera with HA antigen or whole virus and performing the HI assay. The starting dilution is usually 1:10.0. From this dilution, further two-fold dilutions are prepared 1:20.0, 1:40.0, 1:80.0, . . . , 1:2560.0. The HI titer is defined as the dilution factor of that dilution that still completely inhibits hemagglutination, e.g., 10.0, or 20.0, or 40.0, etc. The higher the titer, the more anti-HA antibodies are present.

In a first aspect the invention provides a method for determining the mid-value titer of a substance in a composition. In some methods, the method comprises selecting a series of dilution factors; determining the standard titer of the substance in the composition; and calculating the geometric mean titer between the standard titer and the lowest dilution factor in the series of dilution factors that is greater than the standard titer, to yield the mid-value titer of the substance in the composition. In some methods the substance is an antibody. In some methods the substance comprises at least one anti-hemagglutinin (HA) antibody. In some methods the composition comprises blood or blood serum.

In another aspect the invention provides a method for determining the mid-value titer of a substance in a composition. In some methods, the method comprises selecting a series of dilution factors; determining the standard titer of the substance in the composition; and calculating, using a computer and the following equation, the mid-value titer of the substance in the composition:

log Tₘ=(log Tₛ+log F)/2

in which Tₘ is the mid-value titer, Ts is the standard titer, and F is the lowest dilution factor in the series of dilution factors that is greater than the standard titer. In some methods the substance is an antibody. In some methods the substance comprises at least one anti-hemagglutinin (HA) antibody. In some methods the composition comprises blood or blood serum.

In another aspect the invention provides a method for determining the mid-value titer of a substance in a composition. In some methods, the method comprises selecting a series of dilution factors; determining the standard titer of the substance in the composition; and calculating, using the following equation, the mid-value titer of the substance in the composition:

log Tₘ=(log Tₛ+log F)/2

in which Tₘ is the mid-value titer, Ts is the standard titer, and F is the lowest dilution factor in the series of dilution factors that is greater than the standard titer. In some methods the substance is an antibody. In some methods the substance comprises at least one anti-hemagglutinin (HA) antibody. In some methods the composition comprises blood or blood serum.

In another aspect the invention provides a system for determining the mid-value titer of a substance in a composition. The system may comprise
a memory storage for maintaining a database and a processing unit coupled to the memory storage, wherein the processing unit is operative to select a series of dilution factors; determine the standard titer of the substance in the composition; and calculate the geometric mean titer between the standard titer and the lowest dilution factor in the series of dilution factors that is greater than the standard titer, to yield the mid-value titer of the substance in the composition.

In another aspect the invention provides a system for determining the mid-value titer of a substance in a composition. The system may comprise a memory storage for maintaining a database and a processing unit coupled to the memory storage, wherein the processing unit is operative to select a series of dilution factors;
determine the standard titer of the substance in the composition; and calculate, using the following equation, the mid-value titer of the substance in the composition:

log Tₘ=(log Tₛ+log F)/2
in which Tₘ is the mid-value titer, Ts is the standard titer, and F is the lowest dilution factor in the series of dilution factors that is greater than the standard titer, to yield the mid-value titer of the substance in the composition.

In another aspect the invention provides a computer-readable medium which stores a set of instructions which when executed performs a method for determining the mid-value titer of a substance in a composition. The method executed by the set of instructions may comprise selecting a series of dilution factors; determining the standard titer of the substance in the composition; and calculating the geometric mean titer between the standard titer and the lowest dilution factor in the series of dilution factors that is greater than the standard titer, to yield the mid-value titer of the substance in the composition.

In another aspect the invention provides a computer-readable medium which stores a set of instructions which when executed performs a method for determining the mid-value titer of a substance in a composition. The method executed by the set of instructions may comprise selecting a series of dilution factors; determining the standard titer of the substance in the composition; and calculating, using a computer and the following equation, the mid-value titer of the substance in the composition:

log Tₘ=(log Tₛ+log F)/2

in which Tₘ is the mid-value titer, Ts is the standard titer, and F is the lowest dilution factor in the series of dilution factors that is greater than the standard titer, to yield the mid-value titer of the substance in the composition.

The accompanying drawings, which are incorporated in and constitute a part of this disclosure, illustrate various embodiments and aspects of the present invention. In the drawings:

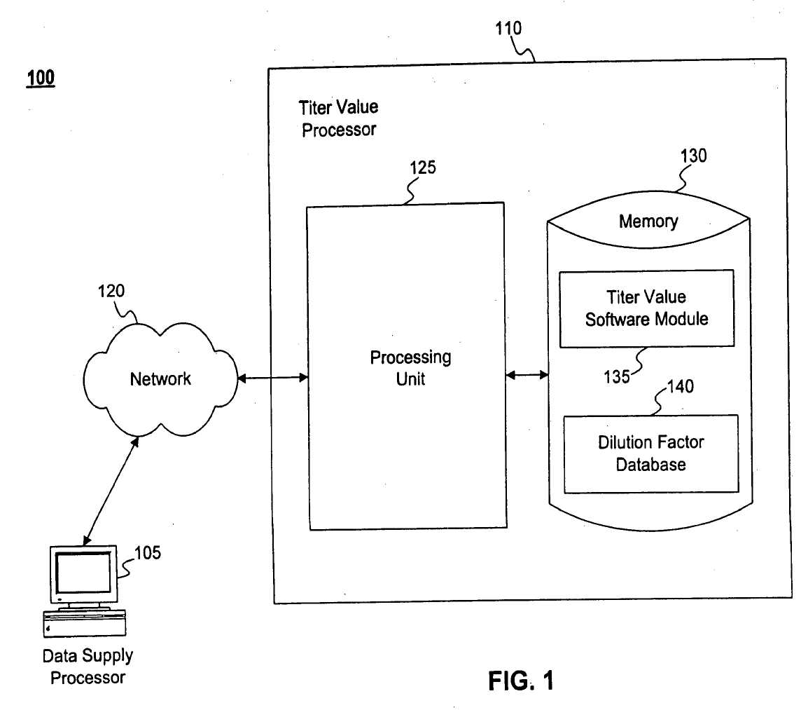

FIG. 1 illustrates a system in which the features and principles of the present invention may be implemented.

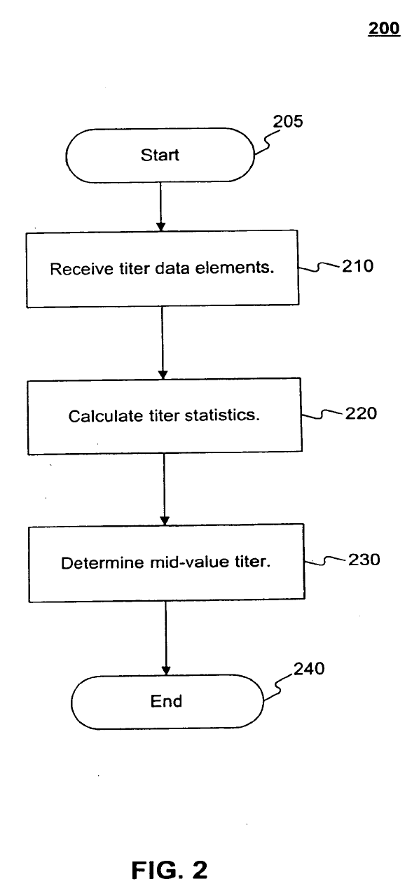

FIG. 2 is a flow chart setting forth the general stages involved in an exemplary method 200 consistent with an embodiment of the invention for determining mid-value titers using, for example, system 100 of FIG. 1.

Herein, titers that are defined as the dilution factor of the highest dilution that still produces a positive or negative test reaction are referred to as “standard titers.” Standard titers are by definition conservative, in the sense that they underestimate the highest dilution that still produces a positive or negative test reaction, i.e., they underestimate the true titer. This bias can be reduced by changing the calculation of titers.

According to the present invention the following calculation can be used:

titer=geometric mean between the standard titer and the lowest dilution factor in the series of dilution factors that is greater than the standard titer

Herein, this is called the “mid-value definition” for titers.

In the present disclosure, “composition” means any form of matter that may contain an analyte, including, for example, solutions, suspensions, blood, blood products, and other bodily fluids. An “analyte” is a substance in the composition the concentration of which is desired. An antibody, such as an anti-HA antibody, is a non-limiting example of an analyte.

Herein, “bias” is used in the statistical sense: what is being measured is on average over- or under-estimated. Standard titers are biased because on average they underestimate the true titers.

By definition, the true titer (Tₜ) lies between the standard titer (Tₛ) and the next dilution factor (F):

Tₛ≦Tₜ≦F

On a logarithmic scale, the mid-value titer (Tₘ) is the mid-point between Tₛ and F:

log Tₘ=(log Tₛ+log F)/2

In almost all practical situations the mid-value definition reduces the bias in the standard titer, meaning that on average

|Tₘ−Tₜ|≦|Tₛ−Tₜ|

In some embodiments of the present invention, three conditions that may be present include that
1. the log-transformed titers are Gaussian distributed or approximately Gaussian distributed;
2. the dilution factors are predefined; and
3. the distance between two consecutive log-transformed dilution factors is small compared to the range of possible log-transformed titer values.
   In still other embodiments, the simultaneous existence of all three conditions may significantly reduce bias in the reported titer.

The mid-value definition may reduce bias in single titers. The mid-value definition is also useful if sets of titers have to be summarized or compared, for example, in reporting many titers measured in clinical trials of, for example, a vaccine.

### EXAMPLE 1

### HI Titer

If the predefined dilution factors are 10.0, 20.0, 40.0, 80.0, 160.0, . . . , 2560.0, and for a specific serum the standard HI titer is 80.0, then the mid-value HI titer is the geometric mean of 80.0 and 160.0, which is 113.1.

### EXAMPLE 2

### HI Titer

For post-vaccination HI titers the three sufficient conditions mentioned above are fulfilled. Log-transformed HI titers are approximately Gaussian distributed; the dilution steps are pre-defined (10.0, 20.0, 40.0, etc.); the distance between two consecutive log-transformed dilution factors (0.7 logₑ[20.0]−logₑ[10.0]=logₑ[40.0]−logₑ[20.0], etc) is small compared to the range of possible log-transformed titer values (logₑ[2560.0]−logₑ[10.0]=5.5.)

Some embodiment consistent with the invention may comprise systems for providing mid-titer values. Such systems may comprise a memory storage for maintaining a database and a processing unit coupled to the memory storage. The processing unit may be operative to receive level data elements, each one of the level data elements corresponding to titer values. In addition, the processing unit may be operative to calculate, using a computer and the following equation, mid-value titers

log Tₘ=(log Tₛ+log F)/2

in which Tₘ is the mid-value titer, Tₛ is the standard titer, and F is the next highest dilution factor above the standard titer. In other words, F is the lowest dilution factor in the series of dilution factors that is greater than the standard titer.

Consistent with another embodiment of the present invention, the aforementioned memory, processing unit, and other components may be implemented in a mid-value titer calculating system. Any suitable combination of hardware, software, and/or firmware may be used to implement the memory, processing unit, or other components. By way of example, the memory, processing unit, or other components may be implemented with any of a data supply processor 105 or a titer value processor 110, in combination with system 100. The aforementioned systems and processors are exemplary and other systems and processors may comprise the aforementioned memory, processing unit, or other components, consistent with embodiments of the present invention.

Furthermore, the invention may be practiced in an electrical circuit comprising discrete electronic elements, packaged or integrated electronic chips containing logic gates, a circuit utilizing a microprocessor, or on a single chip containing electronic elements or microprocessors. The invention may also be practiced using other technologies capable of performing logical operations such as, for example, AND, OR, and NOT, including but not limited to mechanical, optical, fluidic, and quantum technologies. In addition, the invention may be practiced within a general purpose computer or in any other circuits or systems.

By way of a non-limiting example, FIG. 1 illustrates system 100 in which the features and principles of the present invention may be implemented. As illustrated in the block diagram of FIG. 1, system 100 may include data supply processor 105, titer value processor 110, a user 115, and a network 120. User 115 may be an individual, for example, desiring to determine titer values using titer processor 110. User 115 may also be an organization, enterprise, or any other entity having such capabilities.

Titer value processor 110 may include a processing unit 125 and a memory 130. Memory 130 may include a titer value software module 135 and a dilution factor database 140. Software module 135 residing in memory 130 may be executed on processing unit 125, may access database 140, and may implement processes for determining mid-titer values such as any of the methods described herein. Notwithstanding, processor 110 may execute other software modules and implement other processes.

Data supply processor 105 or titer value processor 110 (“the processors”) included in system 100 may be implemented using a personal computer, network computer, mainframe, or other similar microcomputer-based workstation. The processors may though comprise any type of computer operating environment, such as hand-held devices, multiprocessor systems, microprocessor-based or programmable sender electronic devices, minicomputers, mainframe computers, and the like. The processors may also be practiced in distributed computing environments where tasks are performed by remote processing devices. Furthermore, any of the processors may comprise a mobile terminal, such as a smart phone, a cellular telephone, a cellular telephone utilizing wireless application protocol (WAP), personal digital assistant (PDA), intelligent pager, portable computer, a hand held computer, a conventional telephone, or a facsimile machine. The aforementioned systems and devices are exemplary and the processor may comprise other systems or devices.

Network 120 may comprise, for example, a local area network (LAN) or a wide area network (WAN). Such networking environments are commonplace in offices, enterprise-wide computer networks, intranets, and the Internet, and are known by those skilled in the art. When a LAN is used as network 120, a network interface located at any of the processors may be used to interconnect any of the processors. When network 120 is implemented in a WAN networking environment, such as the Internet, the processors may typically include an internal or external modem (not shown) or other means for establishing communications over the WAN. Further, in utilizing network 120, data sent over network 120 may be encrypted to insure data security by using known encryption/decryption techniques.

In addition to utilizing a wire line communications system as network 120, a wireless communications system, or a combination of wire line and wireless may be utilized as network 120 in order to, for example, exchange web pages via the Internet, exchange e-mails via the Internet, or for utilizing other communications channels. Wireless can be defined as radio transmission via the airwaves. However, it may be appreciated that various other communication techniques can be used to provide wireless transmission, including infrared line of sight, cellular, microwave, satellite, packet radio, and spread spectrum radio. The processors in the wireless environment can be any mobile terminal, such as the mobile terminals described above. Wireless data may include, but is not limited to, paging, text messaging, e-mail, Internet access and other specialized data applications specifically excluding or including voice transmission.

System 100 may also transmit data by methods and processes other than, or in combination with, network 120. These methods and processes may include, but are not limited to, transferring data via, diskette, flash memory sticks, CD ROM, facsimile, conventional mail, an interactive voice response system (IVR), or via voice over a publicly switched telephone network.

FIG. 2 is a flow chart setting forth the general stages involved in an exemplary method 200 consistent with an embodiment of the invention for determining mid-value titers using, for example, system 100 of FIG. 1. The titers may comprise, but are not limited to, influenza vaccine titers. Exemplary ways to implement the stages of exemplary method 200 will be described in greater detail below.

Exemplary method 200 may begin at starting block 205 and proceed to stage 210 where processor 110 may receive titer data elements from, for example, from data supply processor 105. Data supply processor 105, for example, may be operated at a medical testing laboratory. The titer data elements, for example, may comprise pre-selected dilution factors and assay read-out data.

From stage 210, where processor 110 receives the titer data elements, exemplary method 200 may advance to stage 220 where processor 110 may calculate one or a plurality of titer statistics. Such titer statistics may include, for example, standard titers determined by converting assay read-out data in light of pre-selected dilution factors.

Once processor 110 calculates the titer statistics in stage 220, exemplary method 200 may continue to stage 230 where processor 110 may determine mid-value titer or mid-value titers if data for more than one titer is received. After processor 110 determines the mid-value titer(s) in stage 230, the mid-value titer(s) may be reported or recorded, and exemplary method 200 may then end at stage 240.

It is to be understood that the foregoing description is exemplary and explanatory only, and should not be considered restrictive of the scope of the invention, as described and claimed. Further, features and/or variations may be provided in addition to those set forth herein. For example, embodiments of the invention may be directed to various combinations and sub-combinations of the features described in the description.

What is claimed is:

1. A method for determining the mid-value titer of a substance in a composition, comprising:
selecting a series of dilution factors;
determining the standard titer of the substance in the composition; and
calculating the geometric mean titer between the standard titer and the lowest dilution factor in the series of dilution factors that is greater than the standard titer, to yield the mid-value titer of the substance in the composition.

2. The method of claim 1, wherein the substance is an antibody.

3. The method of claim 1, wherein the substance comprises at least one anti-hemagglutinin (HA) antibody.

4. The method of claim 1, wherein the composition comprises blood or blood serum.

5. A method for determining the mid-value titer of a substance in a composition, comprising:
selecting a series of dilution factors;
determining the standard titer of the substance in the composition; and
calculating, using a computer and the following equation, the mid-value titer of the substance in the composition:
log Tₘ=(log Tₛ+log F)/2
in which Tₘ is the mid-value titer, Ts is the standard titer, and F is the lowest dilution factor in the series of dilution factors that is greater than the standard titer.

6. The method of claim 5, wherein the substance is an antibody.

7. The method of claim 5, wherein the substance comprises at least one anti-hemagglutinin (HA) antibody.

8. The method of claim 5, wherein the composition comprises blood or blood serum.

9. A method for determining the mid-value titer of a substance in a composition, comprising:
selecting a series of dilution factors;
determining the standard titer of the substance in the composition; and
calculating, using the following equation, the mid-value titer of the substance in the composition:
log Tₘ=(log Tₛ+log F)/2
in which Tₘ is the mid-value titer, Ts is the standard titer, and F is the lowest dilution factor in the series of dilution factors that is greater than the standard titer.

10. The method of claim 9, wherein the substance is an antibody.

11. The method of claim 9, wherein the substance comprises at least one anti-hemagglutinin (HA) antibody.

12. The method of claim 9, wherein the composition comprises blood or blood serum.

13. A system for determining the mid-value titer of a substance in a composition, the system comprising
a memory storage for maintaining a database and a processing unit coupled to the memory storage, wherein the processing unit is operative to:
select a series of dilution factors;
determine the standard titer of the substance in the composition; and
calculate the geometric mean titer between the standard titer and the lowest dilution factor in the series of dilution factors that is greater than the standard titer, to yield the mid-value titer of the substance in the composition.

14. The system of claim 13, wherein the substance is an antibody.

15. The system of claim 13, wherein the substance comprises at least one anti-hemagglutinin (HA) antibody.

16. The system of claim 13, wherein the composition comprises blood or blood serum.

17. A system for determining the mid-value titer of a substance in a composition, the system comprising
a memory storage for maintaining a database and a processing unit coupled to the memory storage, wherein the processing unit is operative to:
select a series of dilution factors;
determine the standard titer of the substance in the composition; and
calculate, using the following equation, the mid-value titer of the substance in the composition:
log Tₘ=(log Tₛ+log F)/2
in which Tₘ is the mid-value titer, Ts is the standard titer, and F is the lowest dilution factor in the series of dilution factors that is greater than the standard titer, to yield the mid-value titer of the substance in the composition.

18. The system of claim 17, wherein the substance is an antibody.

19. The system of claim 17, wherein the substance comprises at least one anti-hemagglutinin (HA) antibody.

20. The system of claim 17, wherein the composition comprises blood or blood serum.

21. A computer-readable medium which stores a set of instructions which when executed performs a method for determining the mid-value titer of a substance in a composition, the method executed by the set of instructions comprising:
selecting a series of dilution factors;
determining the standard titer of the substance in the composition; and
calculating the geometric mean titer between the standard titer and the lowest dilution factor in the series of dilution factors that is greater than the standard titer, to yield the mid-value titer of the substance in the composition.

22. A computer-readable medium which stores a set of instructions which when executed performs a method for determining the mid-value titer of a substance in a composition, the method executed by the set of instructions comprising:
selecting a series of dilution factors;
determining the standard titer of the substance in the composition; and
calculating, using a computer and the following equation, the mid-value titer of the substance in the composition:
log Tₘ=(log Tₛ+log F)/2
in which Tₘ is the mid-value titer, Ts is the standard titer, and F is the lowest dilution factor in the series of dilution factors that is greater than the standard titer, to yield the mid-value titer of the substance in the composition.
